(19) United States
(12) Patent Application Publication
(10) Pub. No.: US 2019/0237181 A1
(43) Pub. Date: Aug. 1, 2019

(54) Method and System for Personalized Injection and Infusion Site Optimization

(71) Applicant: Illume Health LLC, Westport, CT (US)

(72) Inventor: Howard Steinberg, Westport, CT (US)

(73) Assignee: Illume Health LLC, Westport, CT (US)

(21) Appl. No.: 16/260,987

(22) Filed: Jan. 29, 2019

Related U.S. Application Data
(60) Provisional application No. 62/623,457, filed on Jan. 29, 2018.

(51) Int. Cl. G16H 20/17

(52) U.S. Cl. CPC G16H 20/17; G16H 10/60

(57) ABSTRACT

Provided herein are methods and systems for generating dynamic, personalized injection site recommendations. Further provided herein are methods and systems for identifying inconsistencies in medicament absorption and performance at an injection site. Further provided herein are methods and systems for generating a personalized insulin delivery device recommendation.

### BACKGROUND OF THE DISCLOSURE

Some diseases, particularly diabetes, require routine subcutaneous delivery of medicaments, such as insulin, to manage the disease. The regular subcutaneous delivery of medicaments presents a challenge for doctors, patients, and caregivers, as effective disease management ultimately depends on how predictably or consistently the medicament is absorbed and processed by the body. Every patient, however, is different. Variability also exists within individual patients, as a patient may respond differently depending on where on the body the medicament is delivered. This is further complicated by the body's response to the trauma associated with subcutaneous insulin delivery, and sometimes the body's response to delivery of the medicament itself.

While this scenario most frequently arises in the context of patients (and their caregivers) managing insulin-dependent diabetes, it should be appreciated that the underlying concepts are applicable to any treatment plan for any disease or condition that requires the repeated delivery of one or more medicaments beneath the surface of the skin, whether delivered subcutaneously, intramuscularly, or intravenously. The term “patient,” as used herein, refers to the individual receiving treatment and his or her caregivers (i.e., one or more individuals responsible for administering one or more medicaments to the patient). The term “injection,” as used herein, refers to injection by a needle, syringe, pen needle, or the like; the placement of an infusion set or infusion pod for pump devices or other medicament delivery devices; the placement of a subcutaneous monitoring device such as a continuous glucose monitoring (CGM) sensor; or any other action performed that pierces the skin or otherwise causes trauma to underlying tissue as part of a medical treatment plan.

When a patient relies on the subcutaneous delivery of a medicament for disease treatment or management, they are advised to monitor injection sites for any signs of change in the tissue surrounding or underlying the injection site, and discontinue use of an injection site if any changes are seen or palpated. Each time the skin is pierced, the body interprets the injection as a traumatic event, and responds accordingly. In spite of performing these tasks as prescribed by physician and manufacturer, scar tissue may develop, especially when a portion of a medicament delivery device, such as the needle or cannula of an infusion set, or a sensor is left in place and/or moves while inserted, irritating surrounding tissue. Infection may occur as the injection creates a new opening in the skin. Lipodystrophy may also occur, which results in changes in the fatty tissue in the subcutaneous space. Lipoatrophy, a form of lipodystrophy, may result in the breakdown of fatty tissue. Of particular concern to insulin-dependent diabetic patients is lipohypertrophy, which is thought to occur because of the lipogenic nature of the insulin that is delivered subcutaneously, and results in areas of thickened, fibrous, avascular tissue underlying an injection site.

Any change in tissue underlying or surrounding an injection site results will cause the body to absorb the subcutaneously delivered medicament differently and unpredictably, with no way to understand how a perceived or actual change in underlying tissue will impact an individual patient and thus adversely impact glycemic control in diabetes patients. Response varies from patient to patient, varies between anatomical locations in a single patient, and varies from occurrence to occurrence in a single patient. In an attempt to minimize the impact of these changes in underlying tissue and, ultimately, changes in the way a patient responds to delivery of a medicament to a particular site, treatment plans generally stress that the injection sites should be rotated. Rotation allows the body to heal between injections, and reduces the likelihood that relatively small areas of tissue will be continuously exposed to the medicament.

The areas of the body in which subcutaneously delivered medicaments are delivered are illustrated in FIGS. 1(a) (front view) and 1(b) (rear view). Typical injection areas include the abdomen, arms, hip areas, buttocks, and thighs. The term “injection areas,” as used herein, refers to the anatomical locations illustrated in FIGS. 1(a) and 1(b), or any other general area of the body suitable for medicament delivery. The term “injection site,” in contrast, refers to the specific location within an injection area where an injection is delivered. Injection site rotation protocols typically either instruct a patient to move through a series of locations in a specific injection area before proceeding to the next area, or instruct a patient to move through a segment of a sequence of injection areas before moving to the next segment. In some cases, a patient is instructed to follow a specific pattern in one injection area before moving to the next injection area and following a specific pattern. Exemplary injection site rotation patterns are illustrated in FIG. 2. The pattern may vary from injection area to injection area, as different injection areas have different anatomical constraints. In other cases, patients are instructed to use a series of injection areas in sequence, and use a particular segment or part of each injection area and then moving to a corresponding segment of the next injection area in the pattern. For example, a site rotation plan of this type may instruct a patient to use the upper left quadrant or eleven o'clock position of each of a series of injection areas, then use the upper right quadrant or two o'clock position of the same series of injection areas, then use the lower right quadrant or four o'clock position of the same series of injection areas, etc.

Patients are encouraged to track injection sites to facilitate compliance with injection site rotation plans. There is generally poor compliance by patients and there are few, if any, formal tools for managing site rotation. Tracking may occur using traditional paper logging or, in some cases, marking the body itself via the use of templates, temporary tattoos, and the like. As noted above, patients are encouraged to evaluate an injection site before use, visually and via palpation, to determine whether any changes in underlying tissue are detectable. Injection sites where changes are detectable are then skipped in the rotation plan until changes in underlying tissue are no longer detectable or otherwise advised by a health care provider. Absent from these pattern-based injection site rotation plans, however, is any consideration of how an individual patient responds to the delivery of a medicament at a specific injection area or an injection site, whether the response results from changes in absorption of a medicament that may occur before changes in underlying tissue can be palpated or detected visually, or just from the patient's individual anatomy. Every patient is different, and the human body does not always respond predictably. Accordingly, there exists a need for a personalized, adaptable injection site rotation protocol to more effectively manage diseases that require subcutaneous medicament delivery. Additionally, there exists a need to advise patients when use of one or more injection sites is negatively impacting disease management, as well as which sites that patient prefers to use is best next best option.

Some injection areas, such as the arms and abdomen, are also common insertion sites for continuous glucose monitoring sensors (CGM sensors) and flash glucose monitoring sensors (FGM sensors). These sensors monitor glucose levels in interstitial fluid (ISF), which surrounds and feeds the cells of the body. While the insertion of these sensors into the body is traumatic, at least one recent study indicates that sensors that measure glucose in ISF are not impacted by lipohypertrophy. See DeSalvo, D. J., Maahs, D. M., Messer, L., Wadwa, R. P., Payne, S., Ly, T. T., & Buckingham, B. A. (2015). Effect of Lipohypertrophy on Accuracy of Continuous Glucose Monitoring in Patients With Type 1 Diabetes. Diabetes Care, 38(10), e166-e167. Thus, a site that does not exhibit predictable medicament absorption behavior may still be suitable for placement of a sensor that monitors ISF.

### SUMMARY OF THE DISCLOSURE

Provided herein are methods generating a dynamic, personalized injection site rotation plan and notification system. The method comprises obtaining a patient's preferred injection sites; analyzing order and frequency of preferred site utilization; acquiring and analyzing glucose data relating to each of said preferred injection sites; generating a recommendation for a next injection site; and communicating said recommended injection site to the patient. In one implementation, analyzing glucose data includes determining an indication of glycemic control at each of the user's preferred injection sites, and communicating said determination to a user. In another implementation, generating a recommendation comprises removing data concerning a last injection site from the recommendation, and running a multi-armed bandit protocol on the remaining glucose data. In an implementation, the multi-armed bandit protocol is configured to maximize the average time in the desired glucose range. In an implementation, the multi-armed bandit protocol is configured to minimize the variance in glucose readings. In an implementation, the method includes communicating the recommended injection site to a third party. In an implementation, glucose data is blood glucose data. In an implementation glucose data is based on analysis of interstitial fluid. In some implementations, glycemic control at each injection site is analyzed. When glycemic control at each injection site is analyzed, injection sites that do not exhibit sufficient glycemic control may be removed from consideration in the injection site recommendation.

Provided herein are systems for dynamically generating injection site recommendations. The system includes a computing device storing executable instructions in a memory of the computing device; an analyte monitoring device; and a medicament delivery device, and the computing device is configured to receive, as an input, an injection site location, store the injection site location, gather analyte data, correlate data including the analyte data and medicament delivery device, and generate a recommendation for a next injection site location. This recommendation is communicated to the patient. In some implementations, the recommendation for a next injection site location is generated based on a multi-armed bandit protocol. In an implementation, the multi-armed bandit protocol is configured to maximize the average time in a desired range. In an implementation, the multi-armed bandit protocol is configured to minimize the variance in glucose readings or analyte readings. In an implementation, the method includes communicating the recommended injection site to a third party. In an implementation, analyte data is blood glucose data. In an implementation analyte data is based on analysis of interstitial fluid. In some implementations, glycemic control at each injection site is analyzed. When glycemic control at each injection site is analyzed, injection sites that do not exhibit sufficient glycemic control may be removed from consideration in the injection site recommendation.

Provided herein are methods for identifying inconsistencies in medicament absorption and performance at an injection site. The method comprises receiving, as an input, the injection site location, storing the injection site location, gathering analyte data, correlating data including the analyte data and medicament delivery device, and generating an indication of injection sites where the medicament is not absorbed in a predictable fashion. In an implementation, the indication of injection sites where the medicament is not absorbed in a predictable fashion is generated based on analysis of variance techniques.

Provided herein are systems for identifying inconsistencies in medicament absorption and performance at an injection site. The system comprises a computing device storing executable instructions in a memory of the computing device; an analyte monitoring device; and a medicament delivery device, wherein the computing device is configured to receive, as an input, the injection site location, store the injection site location, gather analyte data, correlate data including the analyte data and medicament delivery device, and generate an indication of injection sites where the medicament is not absorbed in a predictable fashion. In an implementation, the indication of injection sites where the medicament is not absorbed in a predictable fashion is generated based on analysis of variance techniques.

Provided herein are methods of generating a dynamic, personalized insulin delivery device recommendation. The method comprises collecting glucose data from a plurality of patients; correlating the glucose data with insulin delivery data, and generating a recommendation for an insulin delivery device based on the correlated data. In an implementation, insulin delivery data includes insulin delivery device, time period, injection site identifier, and/or insulin type/brand. In an implementation, the recommendation is based on analysis of variance techniques performed across each insulin delivery device. In an implementation, the recommendation is an insulin delivery device and injection site pairing. In an implementation, the recommendation of the insulin delivery device, site pairing is based on analysis of variance techniques performed across each insulin delivery device, injection site identifier pair. Any of the methods described herein may be implemented by computing device.

### BRIEF DESCRIPTION OF THE DRAWINGS

FIGS. 1(a) and 1(b) illustrate exemplary injection areas for subcutaneous delivery of medicaments.

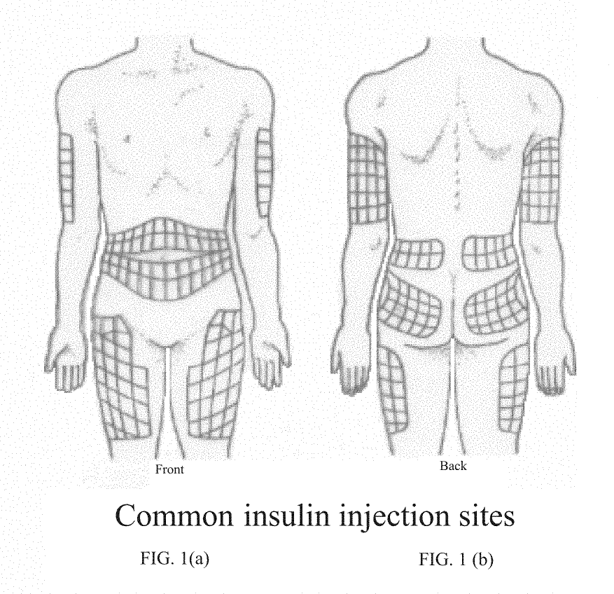

FIG. 2 illustrates exemplary injection site rotation patterns used within an injection area.

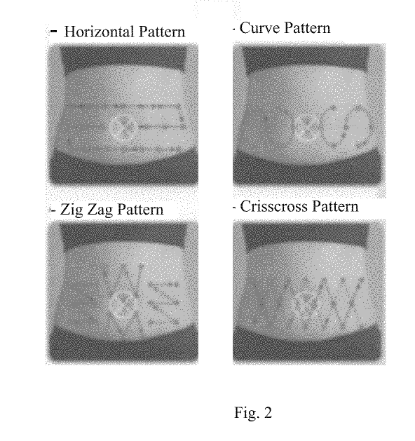

FIG. 3a is a block diagram of an exemplary system for providing a personalized injection site rotation plan, in accordance with the present invention.

FIG. 3b is a diagram of an exemplary system for providing a personalized injection site rotation plan, in accordance with the present invention.

FIG. 4 is a block diagram of an exemplary computing device in accordance with the present invention.

FIG. 5 is a block diagram of an exemplary blood glucose meter in accordance with the present invention.

FIG. 6 is a block diagram of an exemplary continuous glucose monitor in accordance with the present invention.

FIG. 7 illustrates a method for generating an injection site recommendation, in accordance with the present invention.

FIG. 8 illustrates an exemplary method for data collection and collation, in accordance with the present invention.

FIG. 9 illustrates an exemplary method for assessing glycemic control across preferred injection sites.

FIG. 10 illustrates an exemplary method for generating an injection site recommendation, in accordance with the present invention.

FIG. 11 illustrates an exemplary process for generating recommendations relating to insulin delivery devices.

### DETAILED DESCRIPTION OF THE DISCLOSURE

The present invention provides for a method, apparatus, and system for generating a personalized injection site rotation plan for patients in need of subcutaneous delivery of a medicament, and further recommendations regarding associated equipment. The method may be implemented via any known computer technology, for example, as software configured to run on a handheld computing device such as a smartphone, or as software or firmware in a special purpose computing device. In this description, embodiments are described with respect to generating a personalized injection site rotation plan for an insulin-dependent person with diabetes that uses either manual injection via syringe or pen device and/or continuous subcutaneous insulin infusion (CSII) therapy with a continuous glucose monitoring (CGM) system. However, it should be appreciated that the principles described herein do not require a CSII or CGM, and are equally applicable to a patient that relies on multiple daily injection (MDI) therapy and a blood glucose meter (BGM), or a patient that uses a flash glucose monitoring (FGM) system with either SCII, MDI therapy, or a combination of therapies, without departing from the scope of the invention. The various devices used to subcutaneously administer insulin, such as syringes, pen needles, and various types of insulin pumps, are collectively referred to herein as “insulin delivery devices.” Other tools used to measure glycemic control, such as HbA1c levels (commonly referred to as “A1C”) may also be used, either alone or in combination with other quantitative indicators of glycemic control, in generating personalized recommendations without departing from the scope of the invention.

Similarly, it should be appreciated that the methods, apparatus, and systems described herein are applicable to any subcutaneously administered therapy in which the interaction of the administered medicament with the body can be quantitatively measured via, for example, therapeutic drug monitoring techniques. A list of potential analytes is appended to this application. Further, while the method, apparatus, and system described herein make use of data that is acquired automatically and transferred from one device to another via known computer networking and/or data transfer protocols, it should be appreciated that the data may be input to one or more computing devices via manual data entry methods, voice command, or any other known method for inputting data without departing from the scope of the invention.

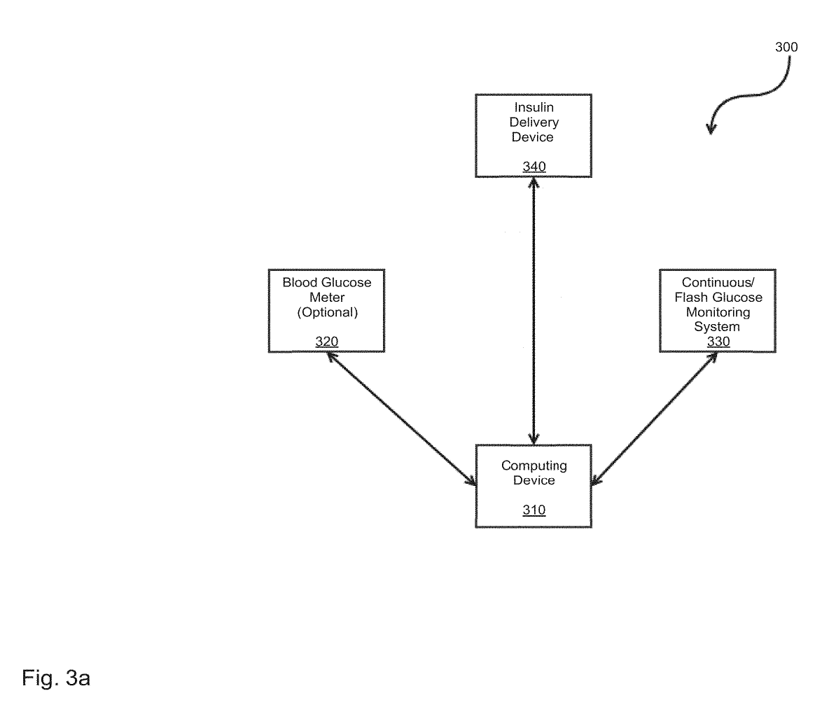

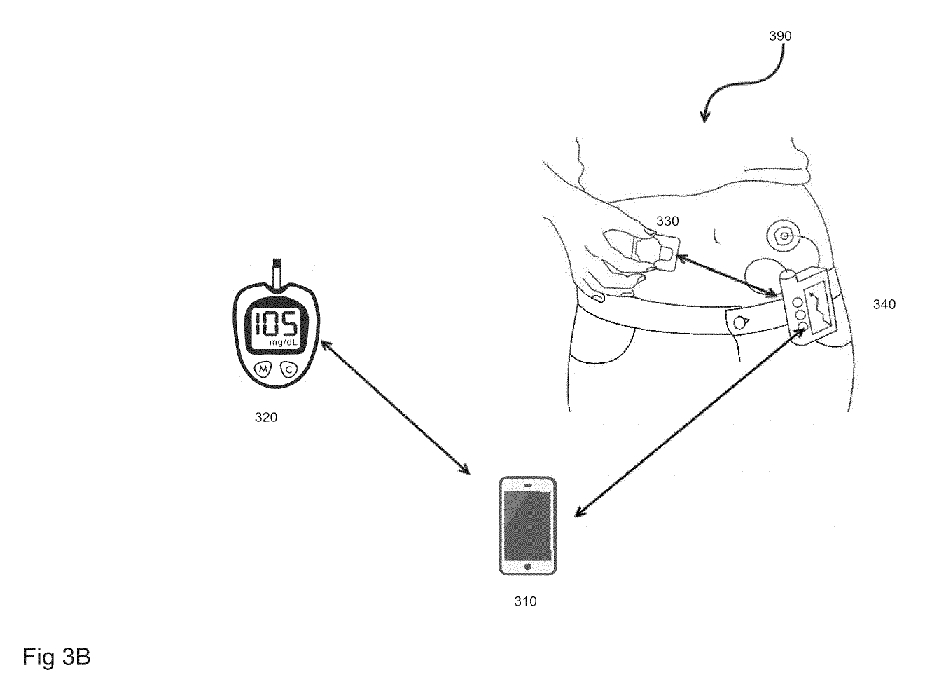

FIG. 3a illustrates an exemplary system for providing a personalized injection site rotation plan 300 in block diagram form, in accordance with the present invention. Exemplary system 300 includes a computing device 310, a blood glucose meter (BGM) 320, a continuous glucose monitoring device (CG) 330, and at least one insulin delivery device 340. FIG. 3b illustrates the same system, including exemplary devices. Computing device 310 may be any computing device that is operable to receive, store, and analyze data, and to provide input/output functionality. Exemplary computing devices include purpose built computing devices, tablet computers, smartphones, notebook computers, laptop computers, and desktop computers. Data may be input by a user via an input device such as a keyboard, touch screen, buttons, or the like. Data may also be transferred from another device, such as a BGM, CGM, another computing device, or a SCII device, via known data transfer protocols including, but not limited to, universal serial bus (USB) serial communications protocols, wired networking protocols, as well as wireless networking and data transfer protocols including RF communications, Bluetooth, WiFi (802.11 protocols), near field communications such as Zigbee, Rubee, and the like. Communication may be bidirectional between devices, or may be unidirectional. Where communication is unidirectional, it is preferable that data be transmitted from the BGM and/or CGM to computing device 310.

The system may further include optional blood glucose meter (BGM) 320 which, if present, is preferably configurable to be in wired or wireless communication with computing device 310. However, as data may be input manually via, for example, buttons, voice command, touch screen, or conventional input/data entry techniques, BGM 320 need not be in communication with computing device 310. Further, in the context of use with a CGM system, BGM 320 is typically used for 1) calibration or 2) when there is reason to believe that CGM results are not reliable. However, it should be appreciated that BGM readings may be used to supplement or in place of glucose data determined via other methods.

The system also includes CGM system 330. CGM system 330 is a convention continuous glucose monitoring device/system and is configured to measure glucose levels in interstitial fluid at regular intervals, for example in 5 minute intervals. Because of the volume of data that is produced by CGM 300, it is preferable that CGM be configured to transfer data to computing device 310, either directly via a wired or wireless communications/data transfer protocol, or indirectly by connecting to a network (via wired or wireless communications/data transfer protocols), uploading CGM data, and providing a conduit for transfer of the CGM data to computing device 310 via, for example, a secure login, VPN tunnel, wired or wireless network connection, application integration, wired or wireless data transfer protocol, or the like. The specifics of data transfer, however, do not limit the present invention in any way. Further, it should be appreciated that, while a CGM is illustrated for exemplary purposes, the system requires at least one source of glucose data. The methods described herein are applicable to any glucose data, whether acquired via BGM, CGM, FGM, or any other means of glucose monitoring, without departing from the scope of the invention. It should be appreciated that a minimum of one source of glucose (or analyte) data is necessary in the system, methods, and apparatus described herein.

Finally, system 300 also includes at least one insulin delivery device 340. While computing device 310 and insulin delivery device 340 may be configured to communicate via wired or wireless communication protocols, for example, to facilitate programming of a CSII device such as an insulin pump, to provide alerts regarding malfunctions or reminders, or the like, this is not required. Insulin delivery device 340 may include a CSII, such as the Omnipod® tubeless insulin pump, MiniMed™ insulin pump system, t:slim X2™ pump system, ACCU-CHEK® combo system, an implantable insulin pump, or the like; an injection device such as a syringe or a pen needle; or a combination of a CSII and one or more injection devices.

It should also be appreciated that functionality of one or more of the devices included in system 300 may be integrated into a single device without departing from the scope of the invention. For example, the CGM system may include an implantable sensor component that communicates wirelessly with a handheld computer that also includes a strip port connector and software configured to measure BGM when a blood glucose test strip is inserted. This handheld computer may also be configured to analyze the combined acquired CGM and BGM data. Data may be stored locally or remotely and transmitted across a local network, wide area network, the Internet, via VPN tunnel, or the like, and may be analyzed locally, via one or more remote computing devices, or via cloud computing resources. Results or determinations made via the methods described herein can be stored locally, on a network storage device, via the Internet, via cloud computing, etc. and can be transmitted via known protocols to specific devices, to specific users other than the patient, and to health care providers or other third parties without departing from the scope of the invention.

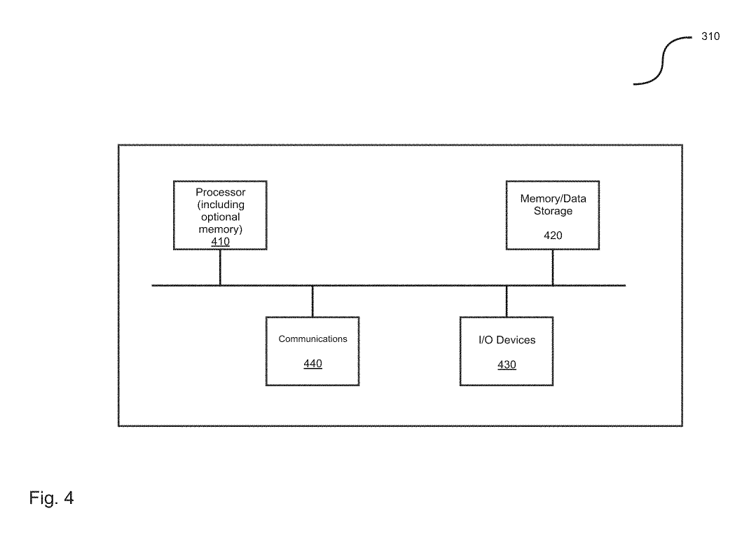

FIG. 4 illustrates an exemplary computing device 310 in accordance with the present invention. As noted above, computing device 310 may be any device that is operable to receive, store, and analyze data, and to provide input/output functionality including, but not limited to, a smartphone, tablet computing device, handheld computing device, personal digital assistant, laptop computer, desktop computer, cloud-connected computing device, or the like. Computing device 310 includes at least one processor 410 and memory 420, Input/Output devices 430, and preferably includes communications devices 440.

Processor 410 executes commands and analyzes data that are stored in memory 420.

Memory 420 stores commands and data. Memory 420 may include a single local storage device, multiple local storage devices, external storage, network-connected storage, cloud-based storage, removable storage, and combinations of these storage devices. Storage devices include all computer readable mediums that can be accessed by processor 410 including both volatile and non-volatile media. Exemplary types of memory used in storage devices include, but are not limited to, random access memory (RAM), read only memory (ROM), electrically erasable programmable ROM (EEPROM), magnetic storage media, flash memory, optical storage such as CD ROM and DVD, or any other computer-readable media for storing information in a non-transitory form.

Input/Output devices 430 are used to provide input to the computing device via, for example, keys, buttons, touch screens, keyboards, touchpads, trackballs, audio (microphone input) and the like; and also used to output information via, for example, a display, speaker, printer, speakers, or by relaying an alert or other output to another device such as a smartwatch. Communications devices 440 include network interfaces, modems, and interfaces that allow computing device 310 to communicate, either directly or indirectly, with other computing devices. This exemplary configuration of computing device 310, however, should not be construed as limiting in any fashion.

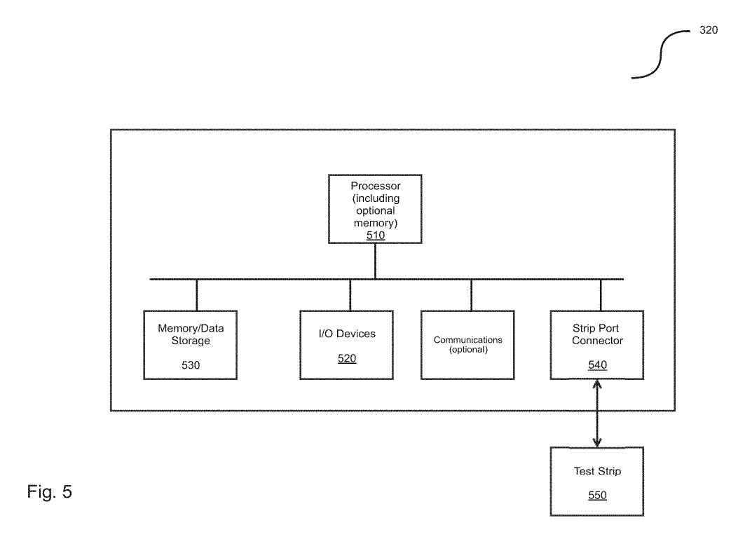

FIG. 5 illustrates an exemplary blood glucose meter 320 in accordance with the present invention. Blood glucose meter 320 includes a processor 510, I/O devices 520, memory 530, and a strip port connector 540, which is configured to receive a biosensor such as test strip 550. Processor 510, I/O devices 520, and memory 530 are as described above with respect to computing device 310. Strip port connector 540 is configured to receive a test strip 550. Memory 530 is configured to store blood glucose readings and, preferably, time stamped blood glucose readings. While an exemplary blood glucose meter is illustrated, it should be appreciated that any device configured to monitor an analyte level, such as glucose, can be substituted without departing from the scope of the invention.

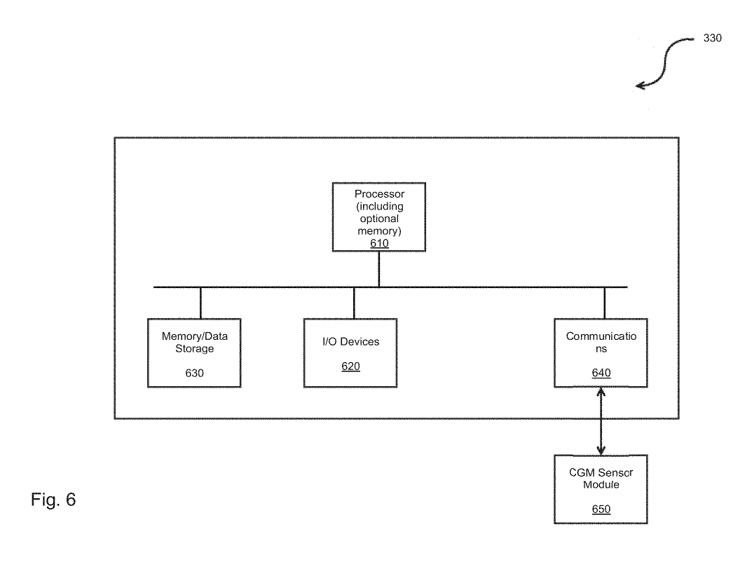

FIG. 6 illustrates an exemplary continuous glucose monitoring system 330 in accordance with the present invention. CGM system 330 is similar to that of BGM 320 in that it includes a processor 610, I/O devices 620, and memory 630. However, rather than include a port for a biosensor, CGM system 330 includes communications means 640 configured to allow communication with a CGM sensor module 650. CGM sensor module 650 provides glucose data to the CGM system 330 at regular intervals via known communications protocols. It should be appreciated that this illustration is only exemplary, and that any analyte monitoring device could be substituted for CGM system 330 without departing from the scope of the invention.

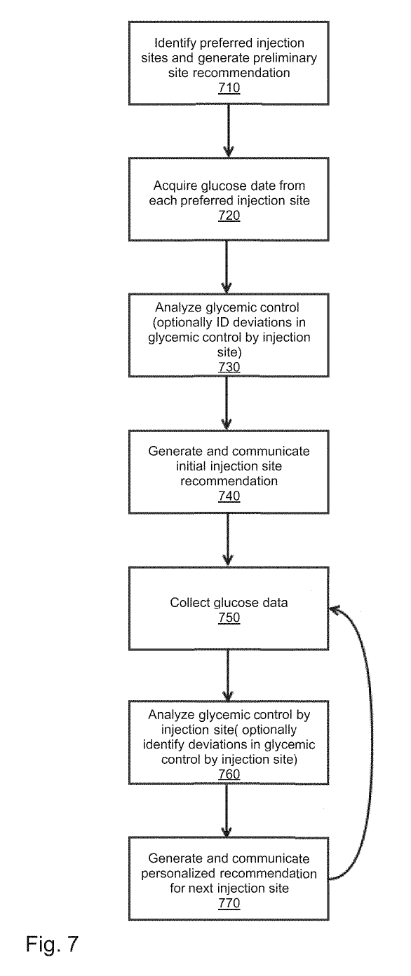

FIG. 7 illustrates a method for generating an injection site recommendation, in accordance with the present invention. The method of FIG. 7 is described herein as being performed by computing device 310. However, it should be appreciated that the method can be performed using other devices, such as a BGM device, a CGM device, a CSII device, or a device that integrates the functionality of one or more of a BGM device, CGM device, and a CSII device.

In the method exemplified in FIG. 7, a patient first identifies preferred injection sites in step 710. Preferred injection sites are those injection sites that are identified by the user as acceptable for injection. These injection sites are typically the injection sites where a patient is most likely to be compliant using based on personal preference. For example, a user may identify preferred quadrants in each of the right arm, left arm, right leg, left leg, and right and left hip areas, reserving the abdominal area for placement of a CGM sensor. Preferred injection sites may be input via a text based interface or a graphical user interface, for example, by using a touchscreen (I/O devices 430) to display the outline of a figure, and identifying each preferred injection site by touching the screen. In other implementations, preferred injection sites may be identified by voice command, or using any other known input device or devices. In some embodiments, the user may prioritize preferred injection sites to set the initial recommended injection site.

A personalized, dynamic site recommendation is generated first on the basis of analyzing all of the preferred injection sites and identifying the least recently used injection site of the preferred set (this would be the “oldest” injection site in preferred set). An initial personalized injection site recommendation can be enhanced by obtaining the glucose data acquired from each of the preferred injection sites, as in step 720. Preferably, this data is timestamped. In the event that existing glucose data is used, in whole or in part, it is preferable that each glucose reading is timestamped and associated with a specific injection site of the preferred injection sites. This information may be added by the patient. In some implementations, the initial round (one rotation through each preferred injection site identified by the patient) of injection sites is based on ordering the preferred injection sites in order of use (or time stamp) beginning with the least recently used injection site and progressing through the list of preferred injection sites in order until the injection site that was most recently used (at the time preferred injection sites are identified) is used. In some implementations, the initial round of rotation may be shuffled by timestamp or randomized. In some implementations, this initial glucose information is stored and used to provide insight on variance of site performance, which is communicated to the patient, without departing from the initial injection site rotation plan. In other implementations, where glucose data is not available for one or more of the preferred injection sites, the personalized injection site rotation plan begins by rotating the patient through those sites that do not yet have data, until all preferred injection sites have data from at least one full lifetime of an infusion set (this varies based on the specific insulin delivery device, but on average is approximately 3 days) or history of manual injections (single injection). Where CGM is used, data should be sampled at the CGM system's normal sampling rate.

Each glucose reading received by computing device 310 is either input by the patient via I/O devices 430, or is received from a BGM, CGM, or another computer system connected via communications device(s) 440. This information is collated by computing device 310 and preferably stored in a database stored in memory 420. It is preferable that each database entry correspond to a glucose reading and include an “insertion” time stamp indicating when the insulin delivery device was inserted or when the last insulin injection occurred, an injection site identifier, and an insulin delivery device type identifier; a glucose reading, and a timestamp associated with the glucose reading. In some implementations, one or more attributes of the database entry, such as injection site identifier, may be associated at a later time, based on, for example, clock data or patient input. Preferably, clocks are synchronized across all devices in system 100. It should be appreciated that additional information, such as demographic and disease information, medical history, and behavioral data regarding how patient engages with their disease may also be stored in this database to further analyze, identify patterns and compare population cohorts.

In step 730, processor 410 analyzes data from each preferred injection site to assess glycemic control at each site. In some implementations, statistical analysis techniques, such as an analysis of variance (ANOVA) techniques are used to determine which injection sites have more variance in glucose readings as compared to other preferred injection sites. More consistency (less variance) in glucose readings when a specific injection site is used is indicative of better glycemic control at that specific injection site. Those sites where variance in glucose readings is statistically significantly higher than other injection sites are also identified as having less than optimal glycemic control. Assessment of glycemic control may be communicated, for example, via an alert on a smartwatch or smartphone, or may be communicated to a third party, such as a health care provide (HCP), via any known messaging or data transfer protocols including, but not limited to, electronic mail, SMS, or the like.

In other implementations, other analysis techniques may be used to assess glycemic control when insulin is introduced at specific sites. For example, a patient may work with a HCP to determine boundaries, ranges, or parameters that provide for optimal glycemic control for a specific patient. In these implementations, statistical analysis methods are used to determine which injection sites provide a patient with acceptable rates of insulin absorption and consistency in absorption over time, and which injection sites are not absorbing insulin in a manner that provides the desired level of glycemic control for that patient.

It should be appreciated that, no matter the analysis employed, the specifics of the analysis performed and parameters used may be customized based on the needs of the patient.

Information regarding the “performance” of injection sites may be communicated to the user via I/O devices 440, as repeated statistically significant variations in glucose readings associated with infusion of a medicament such as insulin a specific injection site may be indicative of changes in underlying tissue—including the build up of scar tissue, lipohypertrophy, lipoatrophy, or changes in vascularization that may warrant further monitoring as part of a self-care routine. In other cases, significant variations in glucose readings may simply indicate that a site is not well-suited for infusion/injection for that specific patient. Isolated occurrences of statistically significant variations in glucose readings at a specific site, however, are not necessarily cause for alarm as numerous other factors may contribute to these variations.

Based on this initial data, an initial injection site recommendation is formulated in step 740 via processor 410. This recommendation is generated using data from all of the preferred injection sites, except for the site where data was most recently acquired. A multi-armed bandit protocol is then run on the data set generated during glycemic control analysis (excluding data from the most recently used injection site). The variable in the multi-armed bandit protocol may be varied depending upon the patient. Exemplary variables include maximizing the average time in range, or minimizing the variance in glucose readings. This recommendation is then communicated to the patient via I/O devices 440.

It should be appreciated that, in some implementations information generated in analyzing glycemic control at specific injection/infusion sites may be used for more than informational purposes, and sites that do not provide sufficient levels of glycemic control, as determined by a HCP or the patient, may be automatically removed from consideration for a period of time, such as one cycle through all injection sites.

In step 750, data is collected from the next injection site, which is preferably the recommended injection site, and stored as described above. However, sometimes external factors render even a preferred injection site inaccessible or unusable, or the preferred injection site may exhibit signs of changes in underlying tissue. In that case, a user can either 1) request a new recommendation, or 2) select the next injection site and specify said site identifier by inputting it via a software interface and input device. If a new recommendation is requested, the recommendation is again generated using the multi-armed bandit protocol, but both the most recent injection site and the original recommendation are removed from consideration.

In some implementations, a patient may also enter information pertaining to the placement of a CGM or FGM sensor. As these sensors measure analyte in interstitial fluid, rather than relying on the absorption of a substance into the bloodstream, they have entirely different concerns regarding their placement. However, as insertion and even continued use of a CGM or FGM sensor may cause trauma and/or irritation and/or a localized immune response, it should be appreciated that, if the location of the sensor is entered, that the method described herein is adaptable to remove the current sensor location from consideration in the determination of the next injection site. In still other implementations, the current sensor location and the previous sensor location are both removed from consideration to allow the sensor insertion site to heal before it is used for infusion or injection.

In step 760, the glucose data is then analyzed—as a whole—to determine which injection sites provide the best glycemic control for the user, and which injection sites may have cause for additional monitoring. In some implementations, deviations in glycemic control are identified by injection site and communicated to the user for informational purposes.

In step 770, a new recommendation is generated using the multi-armed bandit protocol as described in step 740, where the most recently used injection site is excluded in the generation of the next recommendation for an injection site. The data collection process continues. As this process is iterative, recommendations improve as additional data is acquired.

It should be appreciated that, in some implementations, information generated in analyzing glycemic control at specific injection/infusion sites may be used for more than informational purposes, and sites that do not provide sufficient levels of glycemic control, for example, as determined by a HCP or the patient, may be automatically removed from consideration for a period of time, such as one cycle through all injection sites or a period of X number of days.

Preferably, the user interface of the software running on the computing device 310 permits the patient to enter additional information via an input device such as a touch screen, buttons, keyboard, mouse, touchpad, via voice, or the like. Exemplary additional information includes, such as an injection site location if a dataset is not location tagged, or may include data that is to be excluded in making recommendations. For example, if a patient discovers that an infusion set became disconnected from his or her insulin pump during the night, he or she may choose to disregard glucose readings acquired during that time period because no insulin was being administered. Similarly, a patient may change the type of infusion set he or she is using, and then want to reformulate recommendations using only the time periods relating to the new infusion set.

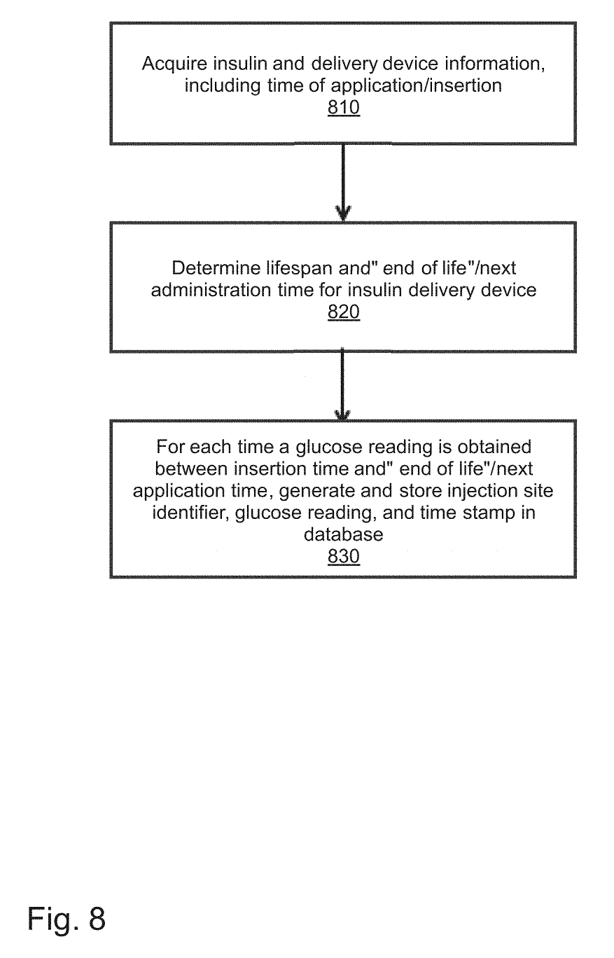

FIG. 8 illustrates an exemplary process for data acquisition and collation in accordance with the present invention. In step 810, information about the insulin delivery device is acquired—either from a user via I/O devices 430 or, in some cases, from the insulin delivery device itself via, for example, a bar code or via wired or wireless communication between computing device 310 and insulin delivery device 340. In some embodiments, the information includes the type/manufacturer of the insulin administered, the device used to deliver insulin, and the time of insertion (in the case of an infusion set) or insulin administration (in the case of a discrete injection of insulin). This information is stored in memory 420 of computing device 430. In step 820, processor 410 executes steps to determine the lifespan of the insulin delivery device (in the case of an infusion set), thus determining when the infusion set should be changed or the next time insulin is to be administered in the case of discrete injections. This information is also stored in memory 420. It should be appreciated that, should an infusion set fail prematurely or an additional injection need to be administered, these parameters may be patient-defined to account for the variations that occur as part of daily life.

In step 830, for each glucose reading that occurs between the insertion time and the calculate the anticipated “end of life” or “next injection” timestamp for the delivery device, a database entry is generated and stored in memory 420. Preferably, the database entry includes the insulin delivery device, the injection site identifier, the glucose reading, and the glucose reading timestamp. This data is then used by processor 410 in the analysis of glycemic control.

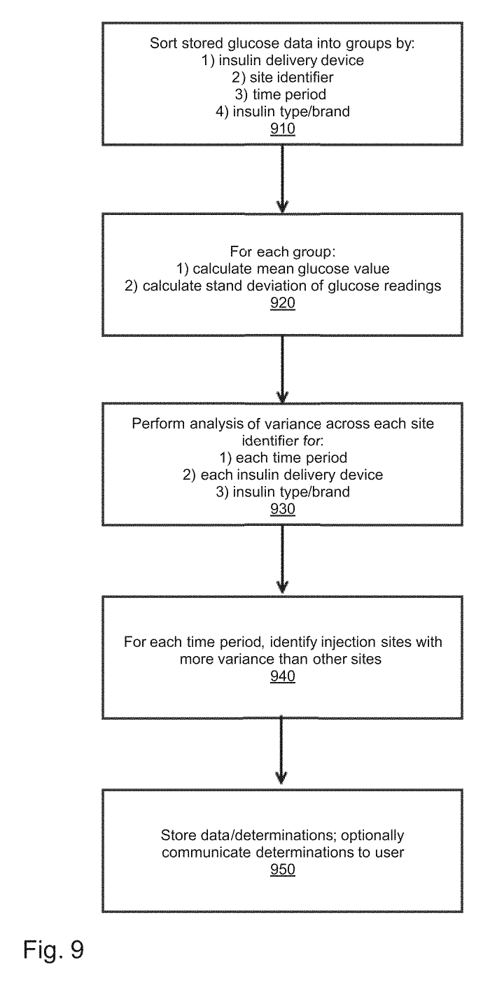

FIG. 9 illustrates an exemplary method for assessing glycemic control across preferred injection sites. Processor 410 of computing device 310 executes the following operations on data stored in memory 420, as described in operation 830.

First, in operation 910, processor 410 sorts the glucose data described in operation 830 into one or more groups based on insulin delivery device, type and brand of insulin, injection site identifier, and the amount of time elapsed since last insertion of infusion set or injection. It should be appreciated that, in different implementations, glucose data may be sorted into groups differently, depending on the factors or variables of interest to the patient and/or the patient's HCP. In operation 920, For each group, processor 410 calculates the mean glucose value and the standard deviation of the glucose readings. It should be appreciated that, while multiple types of glucose readings (blood glucose, continuous glucose, flash glucose) may be used in this determination, conversions and between the various types of glucose readings are known in the art.

In operation 930, the data stored in memory 420 is analyzed using processor 410 for each time period and each device type to determine whether insulin is being absorbed as anticipated and providing the desired level of glycemic control. While specific exemplary methods of analysis are discussed herein, analysis may be adapted based on the needs of a specific patient. In one embodiment, for each time period, each device type, and each insulin type/brand, an analysis of variance (ANOVA) is performed comparing the glucose values across each site identifier to determine which injection sites are associated with more variance in glucose values than other sites. In one embodiment, the ANOVA conducted is a Welch's ANOVA. However, it should be appreciated that other ANOVA techniques may be applied without departing from the scope of the invention. In another embodiment, for each time period and each device type, an F-test for Equality of Variance is conducted to determine whether variance at a site is statistically significantly higher compare to the other preferred injection sites. In another embodiment, site use may be evaluated against defined thresholds. For example, if a site has been used for more than 75% of samples in the last 90 days, it may be flagged as being suspect and removed from the injection site rotation plan for a period of time. In still other embodiments, multiple statistical analysis methods and/or thresholds may be used in combination to identify those injection sites where the patient's body may be absorbing insulin in a less predictable manner, resulting in swings in glucose readings. Further, it should be appreciated that any statistical analysis performed may be adapted to incorporate different factors/variables/concerns, depending upon the needs of the patient and/or the patient's HCP. For example, some patients might only be interested in information regarding an analysis of variance across each site identifier for each time period. The number and type of statistical analyses performed may vary without departing from the scope of the invention.

In operation 940, the results of the analyses in operation 930 are analyzed to determine whether an injection site is resulting in variances in glucose readings that are statistically significant. This indicates that insulin is not being predictably absorbed. In some cases, this may indicate that changes may be occurring in the tissue underlying the injection site that are changing the way that insulin is absorbed, or that a site is overused. In other cases, this may indicate that the site performs atypically. All of these cases may negatively impact glycemic control. Thus, these determinations are useful in selecting future injection sites, and also may be useful to a patient or HCP in better understanding how the patient's body processes the medicament infused or injected at certain sites so that better determinations can be made regarding dosing at different injection sites. The results of the analysis may be communicated to a user via I/O devices 440 so that the patient can follow up with his or her healthcare provider, or may communicated via known messaging and/or data transfer protocols to a third party such as a HCP. In operation 950, these determinations are stored in memory 420, and may optionally be communicated to a patient via an I/O device.

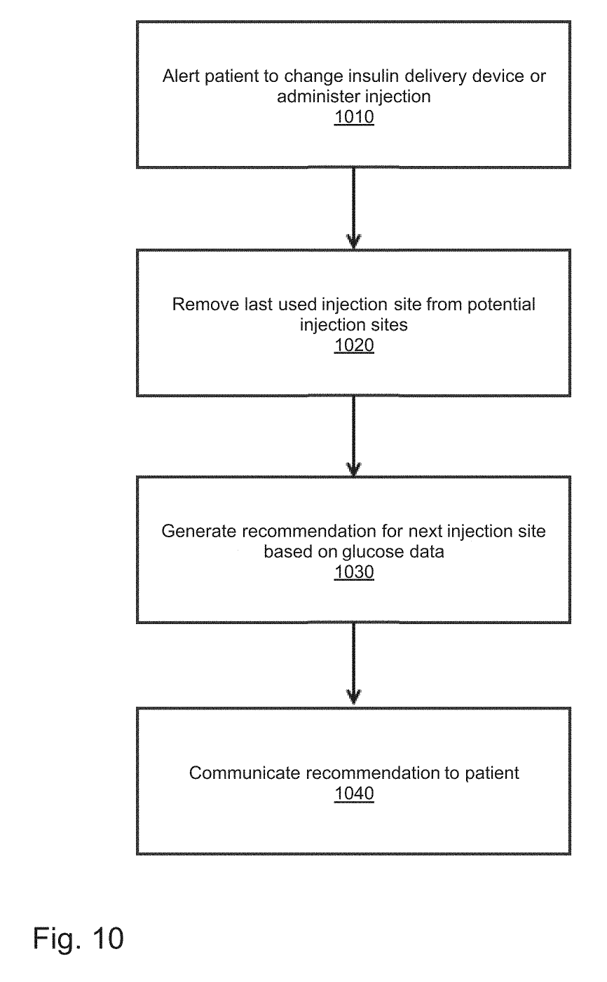

FIG. 10 illustrates an exemplary method for generating an injection site recommendation, in accordance with the present invention. In operation 1010, a patient is alerted to change his or her insulin delivery device (or administer an injection) based on the determination from operation 820. This alert may arrive in any of a variety of forms generated via computing device 310 including, but not limited to, an email, a text or SMS message, an alert pushed to a smartphone to a smartwatch, or simply a text alert on the screen of computing device 310.

In operation 1020, the injection site in use at the time the alert is generated (the last used injection site) is removed from consideration in the list of preferred injection sites, generating a modified list of preferred injection sites. In operation 1030, processor 1030 executes a statistical analysis technique on the glucose data stored in memory 420 to select an injection site from the modified list of preferred injection sites. In some implementations, the statistical analysis technique is a technique such as a multi-armed bandit protocol, which allows for the optimization of a variable in selecting the next injection site. For example, the protocol may be optimized to maximize the average time in range, or minimize the variance in glucose readings. Other techniques may be used without departing from the scope of the invention. Further, as noted above, in some implementations, other injection sites may be removed from consideration based on patient input and/or analysis of glycemic control at each injection site.

In operation 1040, the injection site recommendation is communicated to the user via I/O devices 440 of computer 310. This process is repeated each time an infusion set needs to be changed or an injection needs to be administered. As noted above, when information regarding CGM or FGM sensor placement is available, the current CGM/FGM sensor site or the current CGM/FGM sensor site and the previous CGM/FGM sensor site are removed from the list of preferred injection sites in generating the modified list of preferred injection sites, in order to facilitate healing at those injection sites.

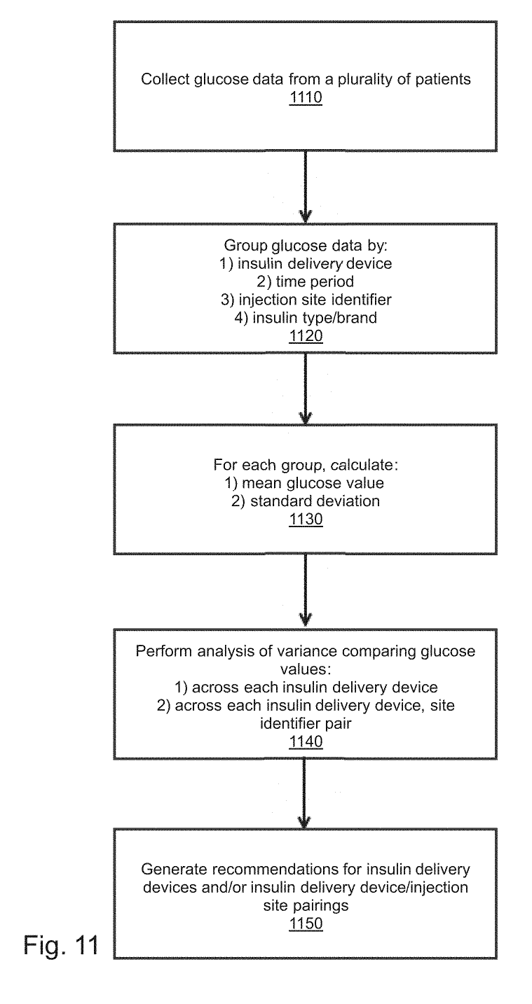

FIG. 11 illustrates an exemplary process for generating recommendations relating to insulin delivery devices. In operation 1110 glucose data, as described in operation 830 of FIG. 8, is collected from a plurality of patients and stored in a database. Preferably, the data is collected via the Internet using known networking protocols between local computing devices and a central computing device or cloud-connected computing system. The computing device then groups the data by insulin delivery device, insulin type and brand, time elapsed since last infusion set change/injection, and injection site identifier in operation 1120. In operation 1130, mean glucose value and standard deviation are calculated for each group. In operation 1140, analysis of variance techniques are applied to the groups of data to determine whether statistically significant variance in glucose values occurs for specific insulin delivery devices or insulins; and whether statistically significant variance in glucose values occurs for specific insulin delivery device, insulins, injection site identifier pairs. This data is stored in the central computing device. In operation 1150, recommendations are generated, based on the results of the analysis of variance, to determine whether some insulin delivery devices or insulins perform better or worse than others, and to determine whether some insulin delivery device/insulin/injection site pairings perform better or worse than others. It should be appreciated that demographic and disease information (without identifying information), as well as information regarding body type, shape, composition, medical history, and the like may also be obtained from the patients using devices and systems in accordance with the present invention, and that such information may be incorporated into the statistical analysis to determine, for example, insulin delivery devices and/or injection sites that may be perform better/worse in a specific population.

Although the invention is described with respect to the analysis of a specific substance (glucose), it should be appreciated that the system and methods described herein can be applied to any analyte, substance, or chemical constituent in a biological flood such as blood, interstitial fluid, cerebral spinal fluid, lymph, urine, or the like, that can be analyzed. These substances may be naturally occurring substances, artificial substances, metabolites, and/or reaction products. It should be appreciated that the specific analyte and analyte sensor/measuring device used do not limit the invention in any way and are, instead, selected based on the analyte to be monitored. Exemplary analytes include lactate; lactic acid; cardiac markers; ketone bodies; acetone; acetoacetic acid; beta hydroxybutyric acid; glucagon, acetyl Co A; intermediaries in the Citric Acid Cycle; choline, testosterone; creatinine; triglycerides; sodium; potassium; chloride; bicarbonate; total protein; alkaline phosphatase; calcium; phosphorus; PO.sub.2; PCO.sub.2; bilirubin (direct and total); red blood cell count; white blood cell count; hemoglobin; hemactocrit; lymphocytes; monocytes; eosinophils; basophils; c-reactive protein; cryoglobulins; fibrinogens; ACTH; aldosterone; ammonia; beta-HCG; magnesium; copper; iron; total cholesterol; low density lipoproteins; high density lipoproteins; lipoprotein A; T4 (total and free); TSH; FSH; LH; ACTH; hepatitis BE antigen; hepatitis B surface antigen; hepatitis A antibody; hepatitis C antibody; acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-.beta. hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, analyte-6-phosphate dehydrogenase, hemoglobinopathies A, S, C, and E, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diphtheria/tetanus antitoxin; erythrocyte arginase; erythrocyte protoporphyrin; esterase D; fatty acids/acylglycines; free .beta.-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; analyte-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17 alpha-hydroxyprogesterone; hypoxanthine phosphoribosyl transferase; immunoreactive trypsin; lactate; lead; lipoproteins ((a), B/A-1, .beta.); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatic virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids may also constitute analytes in certain embodiments. The analyte may be naturally present in the biological fluid, for example, a metabolic product, a hormone, an antigen, an antibody, and the like. Alternatively, the analyte may be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body may also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), and 5-hydroxyindoleacetic acid (FHIAA).

While preferred embodiments of the present invention are shown and described herein, it will be obvious to those skilled in the art that such embodiments are provided by way of example only. Numerous changes, substitutions, and variations will now occur to those skilled in the art without departing from the invention. It should be understood that various alternatives to the embodiments of the present invention described herein may be employed in practicing the invention. The following claims are intended to define the scope of the invention, and cover all methods and systems within the scope of these claims, including their equivalents.

What is claimed is:

1. A method of generating a personalized injection site rotation plan and notification system comprises:
obtaining a patient's preferred injection sites;
analyzing order and frequency of preferred site utilization;
acquiring and analyzing glucose data relating to each of said preferred injection sites;
generating a recommendation for a next injection site; and
communicating said recommended injection site to the patient.

2. The method of claim 1, wherein analyzing glucose data includes determining an indication of glycemic control at each of the user's preferred injection sites, and communicating said determination to a user.

3. The method of claim 1, wherein generating a recommendation for a next injection site comprises removing data concerning a last injection site from the recommendation, and running a multi-armed bandit protocol on the remaining glucose data.

4. The method of claim 3, wherein the multi-armed bandit protocol is configured to maximize the average time in the desired glucose range.

5. The method of claim 3, wherein the multi-armed bandit protocol is configured to minimize the variance in glucose readings.

6. The method of claim 1, wherein glucose data is blood glucose data.

7. The method of claim 1, wherein glucose data is based on analysis of interstitial fluid.

8. A system for generating injection site recommendations, comprising:
computing device storing executable instructions in a memory of the computing device;
an analyte monitoring device; and
a medicament delivery device,
wherein the computing device is configured to receive, as an input, an injection site location, store the injection site location, gather analyte data, correlate data including the analyte data and medicament delivery device, generate a recommendation for a next injection site location, and communicate this recommendation to a patient.

9. The system of claim 8, wherein the recommendation for the next injection site location is generated based on a multi-armed bandit protocol.

10. The system of claim 9, wherein the multi-armed bandit protocol is configured to maximize the average time in a desired range.

11. The system of claim 9, wherein the multi-armed bandit protocol is configured to minimize the variance in analyte readings.

12. A method for identifying inconsistencies in medicament absorption and performance at an injection site, the method comprising:
receiving, as an input, the injection site location;
storing the injection site location;
gathering analyte data;
correlating data including the analyte data and medicament delivery device; and
generating an indication of injection sites where the medicament is not absorbed in a predictable fashion.

13. The method of claim 12, wherein the indication of injection sites where the medicament is not absorbed in a predictable fashion is generated based on analysis of variance techniques.

14. A system for identifying inconsistencies in medicament absorption and performance at an injection site, comprising:
a computing device storing executable instructions in a memory of the computing device;
an analyte monitoring device; and
a medicament delivery device;
wherein the computing device is configured to receive, as an input, the injection site location, store the injection site location, gather analyte data, correlate data including the analyte data and medicament delivery device, and generate an indication of injection sites where the medicament is not absorbed in a predictable fashion.

15. The system according to claim 14, wherein the indication of injection sites where the medicament is not absorbed in a predictable fashion is generated based on analysis of variance techniques.

16. A method for generating a personalized insulin delivery device recommendation, the method comprising:
collecting glucose data from a plurality of patients;
correlating the glucose data with insulin delivery data; and
generating a recommendation for an insulin delivery device based on the correlated data.

17. The method of claim 16, wherein insulin delivery data includes insulin delivery device identifier, time period, injection site identifier, and insulin type/brand.

18. The method of claim 16, wherein the recommendation is based on analysis of variance techniques performed across each insulin delivery device.

19. The method of claim 16, wherein the recommendation generated is an insulin delivery device, and injection site pairing.

20. The method of claim 19, wherein the recommendation of the insulin delivery device, injection site pairing is based on analysis of variance techniques performed across each insulin delivery device, injection site identifier pair.
